(19) United States
(12) Patent Application Publication
(10) Pub. No.: US 2010/0121259 A1
(43) Pub. Date: May 13, 2010

(54) DEVICE AND SYSTEM FOR SKIN TREATMENT

(76) Inventors: Yacov Lutski, Moshav Beit-oved (IL); Revital Berkovitz, Rehovot (IL); Yariv Siman-Tov, Rehovot (IL)

Correspondence Address: NIXON & VANDERHYE, PC, 901 NORTH GLEBE ROAD, 11TH FLOOR, ARLINGTON, VA, 22203, US

(21) Appl. No.: 12/450,503

(22) PCT Filed: Mar. 30, 2008
(86) PCT No.: PCT/IL2008/000442

§ 371 Date: Sep. 29, 2009

Related U.S. Application Data
(60) Provisional application No. 60/908,816, filed on Mar. 29, 2007.

(51) Int. Cl. A61M 35/00

(52) U.S. Cl. 604/22; 604/290

(57) ABSTRACT

A device, system and method for removing pigment from the skin through the application of fluid and vibrations to the skin.

### FIELD OF THE INVENTION

The present invention relates to a device, system and method for the removal of pigment from skin, and in particular, to such a device, system and method which non-invasively removes pigment through the application of fluid and vibrations to the skin.

### BACKGROUND OF THE INVENTION

Unwanted skin pigmentation may take many forms and may arise from many different processes, some of which are natural while other such processes are artificial. A prominent example of an artificial process which results in skin pigmentation is tattooing. This process involves injection of colored pigment into small but deep holes in the skin, typically with needles. Frequently a subject who has a tattoo may wish to remove it. Other types of natural and artificial skin pigmentation may also be unwanted by the subject and so may be desirable to remove.

Pigment removal from skin is currently a difficult process which requires multiple treatments over an extensive period of time. For example, the state of the art treatment involves the use of lasers to remove pigment from the superficial layers of skin. The lasers must be applied at least five times over a period of about 6-8 months, such that the skin has sufficient time to heal between applications. Each application of the laser is painful and results in inflammation and swelling of the skin, accompanied by pain after treatment. The laser cannot remove colors which are similar to skin color, such as yellow or other colors which are not within the spectrum of the laser. Furthermore, side effects may occur, such as burning of the skin, or hypopigmentation (reduced skin color) or hyperpigmentation (increased skin color), all of which are cosmetically undesired.

Other methods of pigment removal include dermabrasion, in which the skin is abraded and hence removed, thereby also removing the pigment. Such treatment is very painful and may cause collateral damage to the skin, potentially resulting in further scarring. Cryosurgery may be used, to freeze a portion of the skin and hence to destroy it, although again this method is very invasive and destructive of the skin. The skin may also be removed by surgery but again this is likely to cause additional scarring and other cosmetic problems.

In an attempt to overcome the drawbacks of known treatments, other methods of pigment removal have been proposed. However, each suffers from many disadvantages. For example, U.S. Pat. No. 6,743,215 to Bernabei relates to a method of dermabrasion, accompanied by mechanical vibrations and the administration of a solution containing some type of pharmaceutical material for treatment of the pigmented area. However, this method suffers from the drawbacks described above, as dermabrasion is highly destructive of the skin and may cause additional scarring.

PCT Publication No. WO2004/009177 to Bernabei relates to the use of electrical pulses in order to cause skin to become permeable, followed by insertion of a solution containing a drug to the permeable skin. This method is suitable for drug delivery but is not suitable for pigment removal, since the skin is caused to become deeply permeable, such that treatment materials would become systemically absorbed. By contrast, pigment removal preferably only involves treatment of the superficial layers of the skin, rather than systemic treatment of the body.

### SUMMARY OF THE INVENTION

There is an unmet need for, and it would be highly useful to have, a system, device and method for removing pigment from the skin which does not cause scarring, skin damage or additional skin complications.

There is also an unmet need for, and it would be highly useful to have, a system, device and method for removing pigment from the skin which is capable of removing pigment of any color without restriction.

There is also an unmet need for, and it would be highly useful to have, a system, device and method for removing pigment from the skin which more rapidly removes the pigmentation, particularly with fewer treatments and/or less time required for recovery of the skin between treatments.

The present invention overcomes these drawbacks of the background art by providing a system, device and method for non-invasively removing pigment from the skin through the application of fluid and vibrations to the skin. Preferably, the fluid is applied under pressure. The vibrations are also preferably at a high rate, up to and including ultrasonic rates. Optionally and more preferably, as described herein, the skin in the area of treatment is caused to protrude, for example through application of a vacuum.

The device preferably comprises a handle for being grasped, from which fluid is emitted, preferably by being sprayed. The handle is also preferably connected to a probe which vibrates and which is capable of applying mechanical vibrations to the area of skin to be treated. Preferably the probe length is between 5 cm to 20 cm while the probe. The handle is manipulated by the user in order to apply the fluid to the area of skin to be treated. The handle is preferably connected to a tube which is in turn connected to a container for containing the fluid. The fluid is pumped out of the container by a pump, which is also connected to the container and/or tube, or both (directly or indirectly). The pump is in turn preferably connected to a power source, which may optionally be a battery or an electrical outlet for example. The fluid in the container may also optionally be warmed or cooled for application.

The distal end of the probe preferably features a tip for being placed on the area of skin to be treated. This tip may optionally enable the user to more steadily and easily center the probe on the area of skin to be treated for example. The tip also preferably provides the vibrations for application to the area of skin to be treated, which are more preferably provided at a high rate, most preferably up to and including ultrasonic rates. Preferably the frequency used is above about 25 kHz and below about 40 kHz and most preferably in the range of 28 kHz to 32 kHz for example 30 kHz. The distal end of the probe is preferably either capable of being sterilized or is disposable. Most preferably the distal end of the probe is 1 mm to 20 mm in diameter or length. Most preferably the probe does not enter the skin while providing a treatment to a specific small area between 1 mm and 20 min.

According to preferred embodiments, the distal end of the probe features one or more outlets for the fluid, most preferably at least two or more fluid outlets. The probe handle also preferably features one or more buttons or other controls for controlling the rate of vibrations, the amount of fluid, whether fluid is applied (if the fluid is not provided continuously), and/or optionally temperature of the fluid, and/or pressure of the applied fluid.

The fluid may optionally and preferably consist of water alone and/or of an aqueous solution. The fluid may also optionally comprise a non-aqueous solution. The fluid may optionally comprise a drug or other treatment material, or a combination of drugs or treatment materials.

The fluid is preferably applied under pressure to the skin. Preferably, the fluid is provided through at least one and more preferably a plurality of fluid jets. The pressure and/or volume of the fluid being administered are each preferably adjustable, for example by providing a plurality of controls to the user. Fluid pressure produced by the jets and applied to the skin optionally and preferably is within the range of 25 to 75′ psig. Most preferably the fluid pressure does not exceed up to about 70 psig. The vibrations are also preferably controllable with regard to frequency, more preferably from about 20 kHz to about 40 kHz. More preferably vibration frequency is controlled to be more than 25 kHz and up to 40 kHz. Most preferably, the vibration frequency is within the range of 28-32 kHz, for example 30 kHz.

According to preferred embodiments of the present invention, there is provided a skin protruder for causing the portion of the skin to be treated to protrude. The protruder preferably lifts the portion the skin away from the tissues below.

The skin protruder preferably comprises a suction device for causing a vacuum to be applied to the portion of the skin to be treated. The device may optionally comprise a cup to be applied to the portion of skin to be treated, connected to a tube which is in turn connected to a pump or other device for inducing suction. For this exemplary embodiment, the previously described probe is preferably inserted through the cup such that the vacuum may be maintained, optionally and preferably through a sleeve which maintains the seal.

Alternatively, a cup or other type of cover may optionally be applied without the application of a vacuum to the skin, for example in order to protect the surrounding skin from contacting the fluid and/or to keep the area of skin to be treated clean and hygienic. Furthermore, such a cover may optionally be applied to protect the user who is applying the treatment from contacting the fluid and/or biological tissue from the subject of the treatment, wherein tissue may comprise liquid and/or solid material.

According to other preferred embodiments of the present invention, if a cover is used (with or without a protruder to cause the skin to protrude), optionally and preferably a suction device is included to remove fluid being applied under the cover. The suction device may optionally be separate from the device for applying a vacuum; either or both may optionally be present on the probe or as separate device(s).

According to still other preferred embodiments of the present invention, there is provided a control center for controlling the frequency of the vibrations, and/or the amount of fluid and/or pressure of the fluid being provided. The control center also preferably enables the user to select the strength of protrusion of the skin (ie the extent to which it protrudes), for example by controlling the amount of vacuum applied to the area of the skin to be treated. These functions of the control center are optionally and preferably implemented through the use of suitable electronic components as is known in the art.

Optionally and preferably, the control center may comprise one or more programmed treatment routines, which may optionally be pre-programmed or alternatively programmed by the user, for automatically performing a particular type of treatment. Optionally, the control center comprises a display as is known and accepted in the art. Optionally, the display provides a user with the ability of selecting a programmed treatment or customizing a treatment routine.

As described herein, the term “pigment” may optionally and preferably include, but is not limited to, pigments which are artificially introduced (for example through tattooing), pigmentation which is the result of wound healing or scarring (or any other damage to the skin), or any naturally occurring pigmentation, including but not limited to age spots, freckles, port wine stains and other birthmarks, and the like.

Without wishing to be limited by a single hypothesis, the present invention permits the use of less aggressive treatment than methods of the background art. In turn, this permits the skin to heal more rapidly. Without wishing to be limited in any way, it is believed that the present invention has at least the following advantages: ability to remove any type of pigment without being limited by pigment color or any other limitation; non invasive; reduced likelihood of scarring; reduced time between successive treatments (due to shortened skin healing time); reduction of time from initial removal until complete disappearance; optional use of acid or other treatment solutions having low concentrations (thereby reducing the potential for side effects); parallel control of pulse frequency and pressure, and application of the fluid; optionally enclosing the working area (through the application of a cup or other cover) such that the surrounding skin does not come into contact with the solution/acids and also preventing the user of the device from coming into contact with biological liquids and/or solids; keeping the working area sterile and hygienic through the optional use of an enclosure for the working area; ability to change the profile of the fluid application according to the location of the treatment area; increased ease of use, including also for low skill or non-skilled users; low cost, particularly as compared to laser treatment.

The present invention differs from the solutions proposed in the art in many ways. One non-limiting example of such a difference relates to spraying fluid under pressure. Another non-limiting example of such a difference is the avoidance of needles for puncturing the skin and/or other invasive methods.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or stages manually, automatically, or a combination thereof. Moreover, according to actual instrumentation and equipment of preferred embodiments of the method and system of the present invention, several selected stages could be implemented by hardware or by software on any operating system of any firmware or a combination thereof. For example, as hardware, selected stages of the invention could be implemented as a chip or a circuit. As software, selected stages of the invention could be implemented as a plurality of software instructions being executed by a computer using any suitable operating system. In any case, selected stages of the method and system of the invention could be described as being performed by a data processor, such as a computing platform for executing a plurality of instructions.

It should be noted that optionally any device featuring a data processor and/or the ability to execute one or more instructions may be described as a computer, including but not limited to a PC (personal computer), a server, a minicomputer, a cellular telephone, a smart phone, a PDA (personal data assistant) or a pager.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A-B are a schematic diagrams of optional embodiments of a system according to the present invention, FIG. 1A according to a first exemplary embodiment and FIG. 1B according to a second exemplary embodiment.

FIG. 2 is a schematic diagram of a system according to the present invention according to a second exemplary embodiment.

FIG. 3A-E are schematic diagrams of bottom views of the probe's distal face according to optional embodiments of the present invention.

FIG. 4A-I are schematic diagrams of the probe and treatment area according to an optional embodiments according to the present invention.

FIG. 5 shows a flowchart of an exemplary, non-limiting method according to the present invention for pigment removal.

FIG. 6 is a photograph showing all eight sites after tattooing (introduction of pigment) prior to treatment according to an optional embodiment of the present invention.

FIG. 7 is a photograph showing four sites following one treatment according to the present invention.

FIG. 8 is a photograph of two of the four sites of FIG. 7 following a second treatment according to the present invention.

FIG. 9 is a photograph showing the entire area of skin as in FIG. 6 after the second treatment was applied according to an optional embodiment of the present invention.

FIG. 10A-C depicts close up comparative views of before and after treatment according to an optional embodiment of the present invention.

FIG. 11 A-D are photographs of histological slides of the skin following treatment according to an optional embodiment of the present invention.

### DESCRIPTION OF THE PREFERRED EMBODIMENTS

The present invention is of a system, device and method for removing pigment from the skin through the application of fluid and vibrations to the skin. The fluid may optionally and preferably be applied continuously or alternatively may optionally be provided discontinuously or intermittently, optionally and preferably according to manual control. The device preferably comprises a handle for being grasped, from which fluid is emitted. The handle is manipulated by the user in order to apply the fluid to the area of skin to be treated, wherein the fluid is preferably under pressure. Most preferably the fluid is pumped under pressure, optionally providing a fluid pressure from about 25 psig to about 75 psig, most preferably the fluid pressure is about 70 psig. The handle is preferably connected to a probe which vibrates and which is capable of applying mechanical vibrations to the area of skin to be treated. The vibrations are preferably at a high rate, more preferably up to and including ultrasonic rates. Preferably, the vibration frequency is more than about 25 kHz and below about 40 kHz, most preferably in the range of about 28 kHz to about 32 kHz, providing for a probe that vibrates at a frequent of about 30 kHz for example. Optionally the probe is between 5 cm and 20 cm in length. The handle is also preferably connected to a tube which is in turn connected to a container for containing the fluid. The fluid is pumped out of the container by a pump, which is also connected to the container and/or tube, or both (directly or indirectly). Most preferably the fluid is pumped under pressure, optionally providing a fluid pressure from about 25 psig to about 75 psig; optionally and most preferably the fluid pressure is about 70 psig. The pump is in turn preferably connected to a power source, which may optionally be a battery or an electrical outlet for example.

The distal end of the probe preferably features a tip for being placed on the area of skin to be treated. This tip may optionally enable the user to more steadily and easily center the probe on the area of skin to be treated for example. Optionally, the probe tip is optionally 1 mm to 20 mm in diameters. The tip also provides the vibrations for application to the area of skin to be treated. The distal end of the probe is preferably either capable of being sterilized or is disposable.

According to preferred embodiments, the distal end of the probe preferably features one or more outlets for the fluid. The handle also preferably features one or more buttons or other controls for controlling the fluid amount, whether fluid is applied (if not applied continuously) and/or pressure of the fluid, as well as preferably enabling control of the rate of vibrations.

The fluid may optionally and preferably consist of water alone and/or of an aqueous solution. The fluid may also optionally comprise a non-aqueous solution. The fluid may optionally comprise a drug or other treatment material, or a combination of drugs or treatment materials. The drug or treatment material may optionally comprise one or more of an alpha hydroxy acid or a beta hydroxy acid, including but not limited to one or more of salicylic acid, glycolic acid or lactic acid, or a combination thereof.

The fluid is preferably applied under pressure to the skin. Preferably, the fluid is provided through at least one and more preferably a plurality of fluid jets. The pressure and/or volume and/or optionally the temperature of the liquid are each preferably adjustable, for example by providing a plurality of controls to the user.

According to preferred embodiments of the present invention, there is provided a skin protruder for causing the portion of the skin to be treated to protrude. The protruder preferably lifts the portion the skin away from the tissues below. Without wishing to be limited by a single hypothesis, it is believed that lifting the skin causes it to become stretched, hence rendering it more permeable or at least sensitive to the treatment. With such stretching, it is believed that one or more treatment parameters may be altered, preferably through reduction, including but not limited to reducing the length of each treatment, the total number of treatments, the pressure for each pulse, the frequency of pulses, or the concentration of the drug or other treatment material included in the solution (if any), or a combination thereof.

The skin protruder preferably comprises a suction device for causing a vacuum to be applied to the portion of the skin to be treated, which may optionally be located on the probe or which alternatively may optionally be separate from the probe. The device may optionally comprise a cup to be applied to the portion of skin to be treated, connected to a tube which is in turn connected to a pump or other device for inducing suction. For this exemplary embodiment, the previously described probe is preferably inserted through the cup such that the vacuum may be maintained, more preferably with a sleeve for maintaining the vacuum. Optionally and preferably, the cup features a seal for sealing the edge of the cup to the skin.

Alternatively, a cup or other type of cover may optionally be applied without the application of a vacuum to the skin, for example in order to protect the surrounding skin from contacting the fluid and/or to keep the area of skin to be treated clean and hygienic. Furthermore, such a cover may optionally be applied to protect the user who is applying the treatment from contacting the fluid and/or biological tissue from the subject of the treatment.

According to other preferred embodiments of the present invention, if a cover is used (with or without a protruder to cause the skin to protrude), optionally and preferably a suction device is included to remove fluid being applied under the cover. The suction device may optionally be separate from the device for applying a vacuum.

For either embodiment, the cup or cover is preferably transparent or translucent for viewing the area of skin to be treated.

According to still other preferred embodiments of the present invention, there is provided a control center for controlling the frequency of the vibrations, and/or the amount of fluid and/or pressure and/or optionally the temperature of the fluid provided. If more than one such fluid is applied (for example, a fluid containing a treatment material and another fluid which does not contain such a material), then preferably the control center allows the user to select the rate of application of each fluid separately. The control center also preferably enables the user to select the strength of protrusion of the skin (ie the degree to which it protrudes), for example by controlling the amount of vacuum applied to the area of the skin to be treated. These functions of the control center are optionally and preferably implemented through the use of suitable electronic components as is known in the art.

Optionally and preferably, the control center may comprise one or more programmed treatment routines, which may optionally be pre-programmed or alternatively programmed by the user, for automatically performing a particular type of treatment. For example, one or more routines may optionally be provided for different types of applications of fluid according to the nature of the pigment (for example, whether it is a tattoo or a naturally occurring pigmentation) and/or the nature of the skin of the user (thick or thin, sun damaged, young or old, etc) and/or according to the number of the treatment (ie whether the treatment is the first treatment, second treatment and so forth) and/or according to the response of the skin to a previous treatment. Such programmed routines may optionally be programmed onto some type of processor and memory, optionally in some type of computational device.

The principles and operation of the present invention may be better understood with reference to the drawings and the accompanying description.

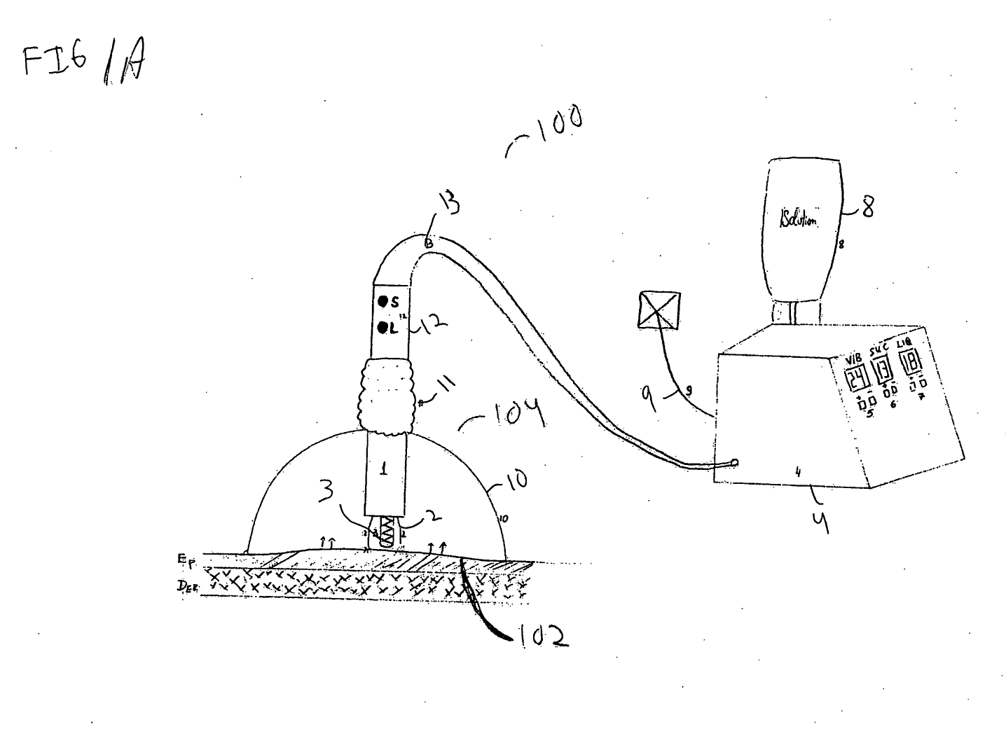

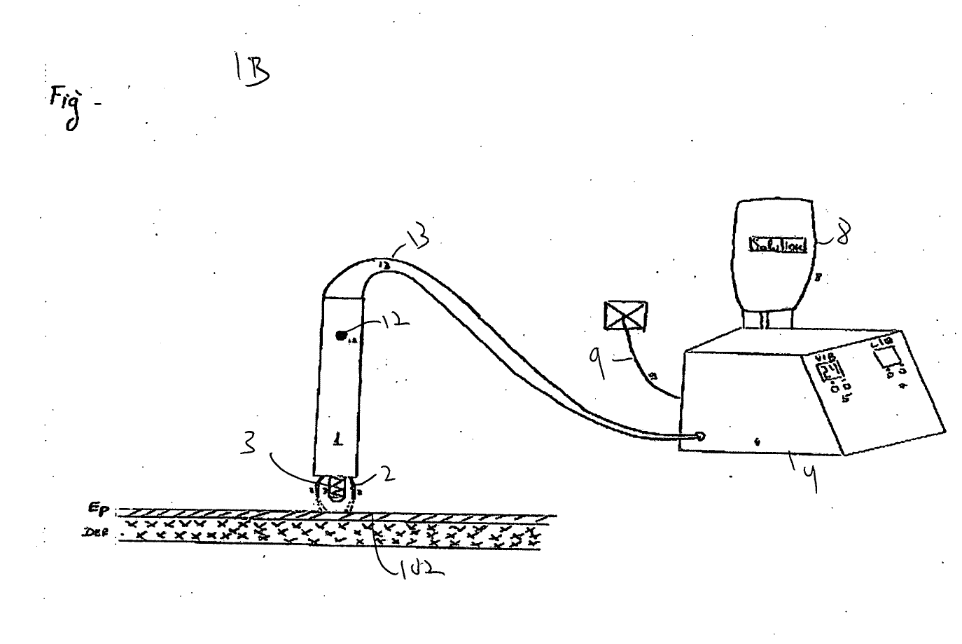

Referring now to the drawings, FIG. 1 is a schematic diagram of a system according to the present invention according to a first exemplary embodiment. A system 100 features a probe 1, which preferably delivers fluid to the area of skin to be treated 102, for example through one or more tubes (not shown). If present, optionally suction is also delivered through probe 1, again for example through one or more tubes (not shown).

The distal end of probe 1 preferably features one or more fluid outlets 2, of which two are shown for the purpose of illustration only and without any intention of being limiting. If two such outlets 2 are present, preferably each outlet 2 is disposed on an opposing side of the distal end of probe 1. If more than two such outlets 2 are present, preferably outlets 2 are disposed evenly around the distal end of probe 1. Each fluid outlet 2 preferably sprays fluid onto skin area 102 for treatment. The size of fluid outlet 2 is optionally and preferably determined according to the desired energy of the fluid to be applied, which in turn is dependant upon a combination of parameters, including but not limited to, the diameter of fluid outlet 2, the pressure of spraying of the fluid and the distance from fluid outlet 2 to skin area 102. Each fluid outlet 2 may also optionally comprise a plurality of holes (or optionally protrusions) for emitting the fluid, preferably having a configuration for altering the pressure or velocity of fluid as emitted.

The distal end of probe 1 preferably also features a probe head 3. Probe head 3 may optionally be made from metal or any other sterilizable material. Alternatively or additionally, probe head 3 may optionally and preferably be disposable. Probe head 3 may optionally be placed in contact with skin area 102, for example to enable the user (not shown) to more readily maintain contact with skin area 102 and/or to ensure proper placement of the distal end of probe 1. Probe head 3 may also optionally itself vibrate at a rate of from about 20 kHz to about 40 kHz.

Probe head 3, as described in greater detail below, may optionally and preferably vibrate due to vibrations from a vibration source (not shown), optionally and more preferably according to a piezoelectric effect (or rather transduction of such an effect). Such a general effect is known in the art for dental tools for cleaning teeth, for example. U.S. Pat. No. 4,038,571 to LITTON INDUSTRIAL PRODUCTS, issued on Jul. 26, 1977 and hereby incorporated by reference as if fully set forth herein, describes a device which uses a piezoelectric crystal for converting electrical energy into mechanical vibrations which are transmitted to a dental workpiece for use in cleaning teeth. As described in the patent, a piezoelectric material, such as a crystal for example (a non-limiting example of which is a lead zirconate-lead titanate ceramic crystal), is supplied with electrical energy. The piezoelectric material then vibrates according to the amount and/or frequency of energy supplied and the inherent properties of the piezoelectrical material itself. The vibrations of the piezoelectric material then causes probe head 3 to vibrate, either directly or through some type of connector or transducer as described in greater detail below.

The function of system 100 is preferably controlled through a control center 4, which contains a pump for pumping the fluid through probe 1 to each outlet 2 (not shown). Control center 4 optionally and preferably includes a pump for providing suction (also not shown), as well any electrical components for controlling the function of system 100 as described herein. The fluid itself is preferably contained in a container 8, which is fluidly connected to probe 1 through control center 4, for being pumped out by the pump in control center 4. The fluid in container 8 may optionally be warmed or cooled as is known in the art.

Control center 4 also preferably features a frequency control switch 5 which more preferably includes a digital display and control, for the controlling the frequency of vibration of probe head 3. Probe head 3 is optionally caused to vibrate according to any mechanism or means that are known in the art, preferably according to known ultrasonic mechanisms or means.

Control center 4 also optionally and preferably features a suction control switch 6 including a digital display and control, for controlling suction applied to skin area 102 as described in greater detail below.

Control center 4 also optionally and preferably features a fluid flow control switch 7 including a digital display and control, for controlling the volume and/or pressure of fluid to be delivered.

Control center 4 features a power source 9, shown herein as an electrical cable for being connected to an electrical outlet, although of course any suitable power source may optionally be used for powering the pump(s) and controls of system 100.

System 100 preferably features a connector 13 for connecting probe 1 to control center 4. Connector 13 preferably includes a tube for carrying fluid to probe 1, an electrical connection for connecting probe 1 to control center 4, and also may optionally include a tube for providing suction to probe 1. Probe 1 also preferably features at least one control button or switch 12, for enabling the user (not shown) to control at least one function of probe 1. As shown an “L” button preferably enables the user to control whether liquid is being sprayed from outlet(s) 2 or not. Each control button 12 is preferably electrically connected to control center 4 through connector 13, for example for turning a pump on or off, or for opening/closing a valve for permitting fluid to flow through connector 13 to probe 1 (not shown).

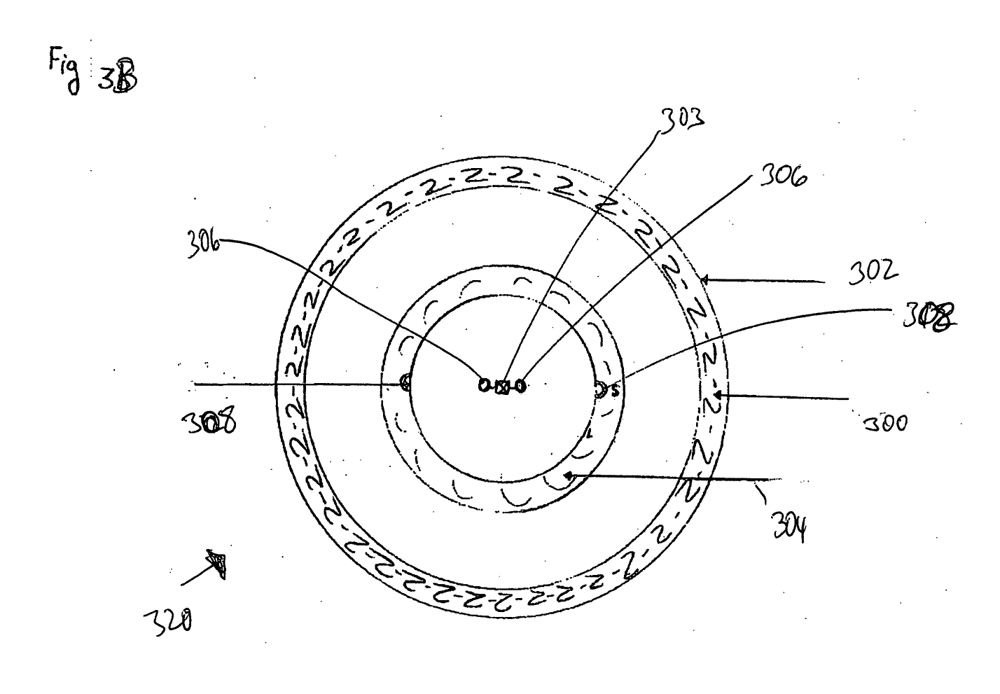

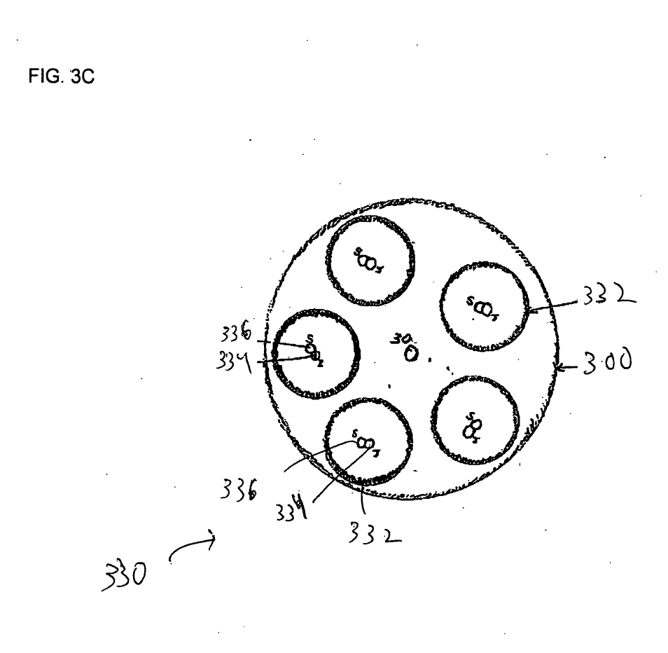

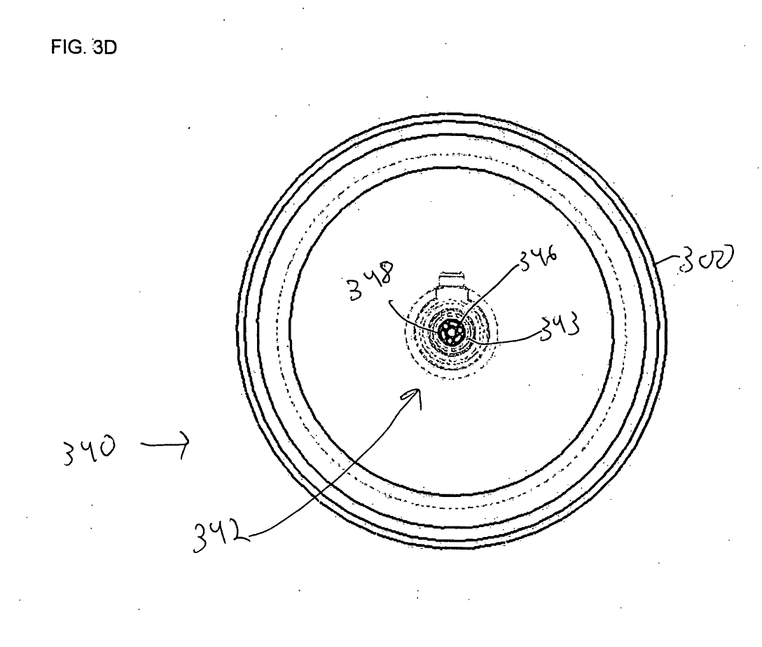

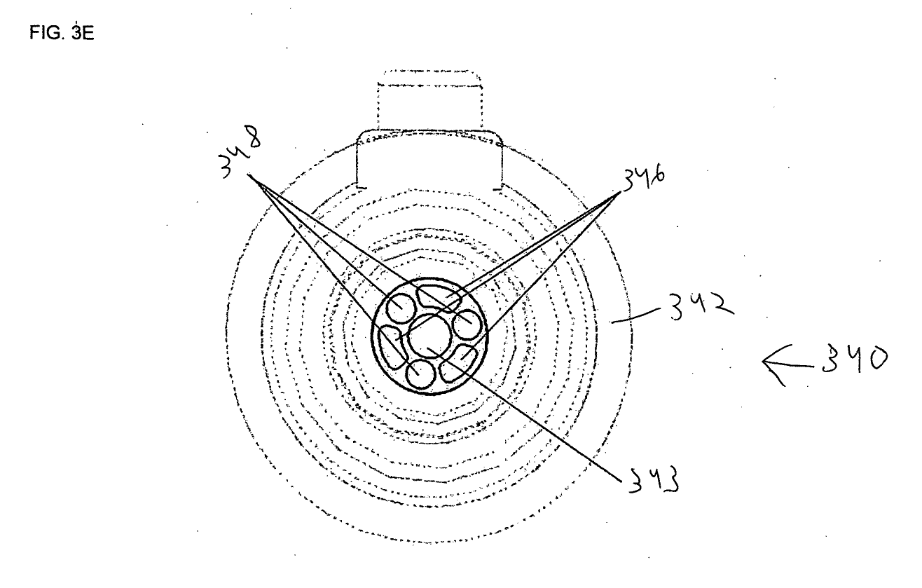

According to preferred embodiments of the present invention, system 100 features a protruder 104 for causing skin area 102 to protrude. Protruder 104 is preferably used to cause skin area 102 to be stretched or pulled; without wishing to be limited by a single hypothesis, it is believed that such stretching increases the sensitivity and/or permeability of the skin to treatment. Protruder 104 preferably features the application of vacuum through a cover 10, which may optionally be shaped like a cup or dome as shown. Cover 10 is preferably transparent or at least translucent, in order for the user to be able to view skin area 102, and may be made from any suitable material, including but not limited to glass, plastic, silicon, rubber, flexible composite materials and the like. Cover 10 preferably features a seal for sealing the edge of cover 10 to skin area 102 (not shown, see FIG. 3).

For this embodiment, system 100 also preferably features a sealing sleeve 11 through which probe 1 is inserted in order to provide a seal for the vacuum and to maintain the vacuum. Sleeve 11 is preferably made of a flexible material such as rubber or plastic to permit probe 1 to be maneuvered. More preferably, sleeve 11 provides a working angle of 60-120 degrees. Sleeve 11 may optionally be configured as an accordion or bellows unit of pleated material, for example.

Also for this embodiment, probe control button 12 also preferably includes a button for controlling suction, labeled “S” in FIG. 1 for the purpose of illustration only and without any intention of being limiting.

Optionally and preferably, in addition to vacuum, the distal end of probe 1 system 100 may also include suction for removing fluid sprayed from outlet(s) 2 (see FIG. 3). Such suction may be applied in addition to the vacuum or in place of the vacuum, for example if cover 10 is used but without the application of a vacuum to skin area 102. Cover 10 may optionally be used without vacuum to maintain cleanliness and hygiene of the area, to prevent fluid from contacting skin other than at skin area 102, and also to protect the user who is operating system 100 from contacting the fluid and/or biological tissue.

FIG. 1B is a schematic diagram of a system according to the present invention according to a second exemplary embodiment. Components with the same numbers have the same or similar function as shown with regard to FIG. 1A; hence the function of the system of FIG. 1B is very similar if not identical, except that there is no device for applying suction or a vacuum. There is also no button “S” for controlling suction; rather button 12 controls whether fluid is sprayed or not.

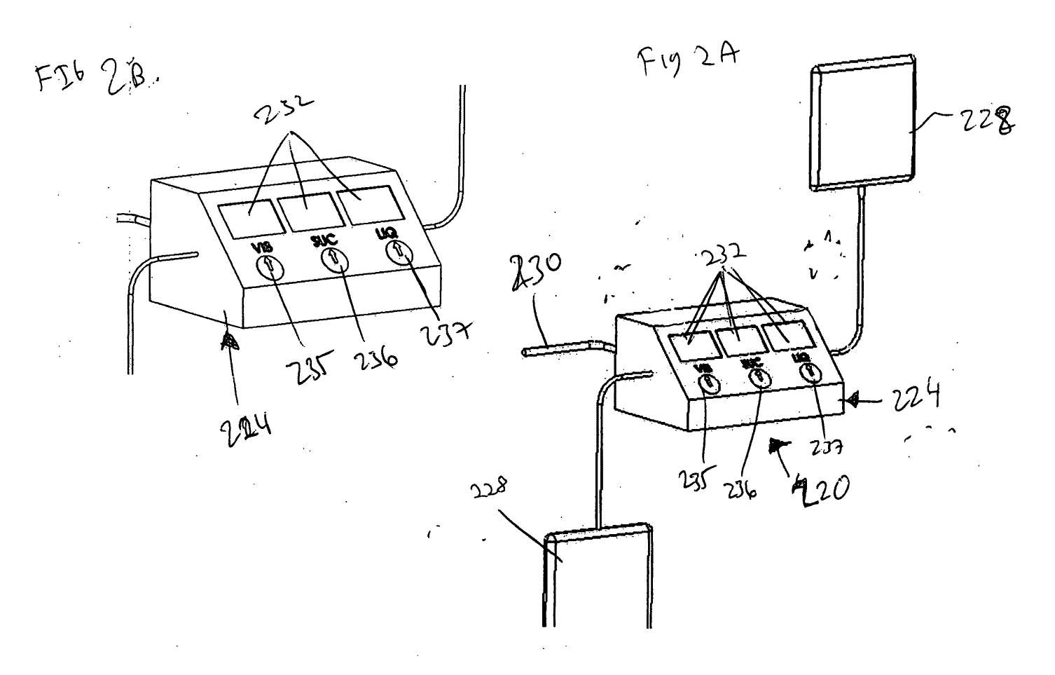

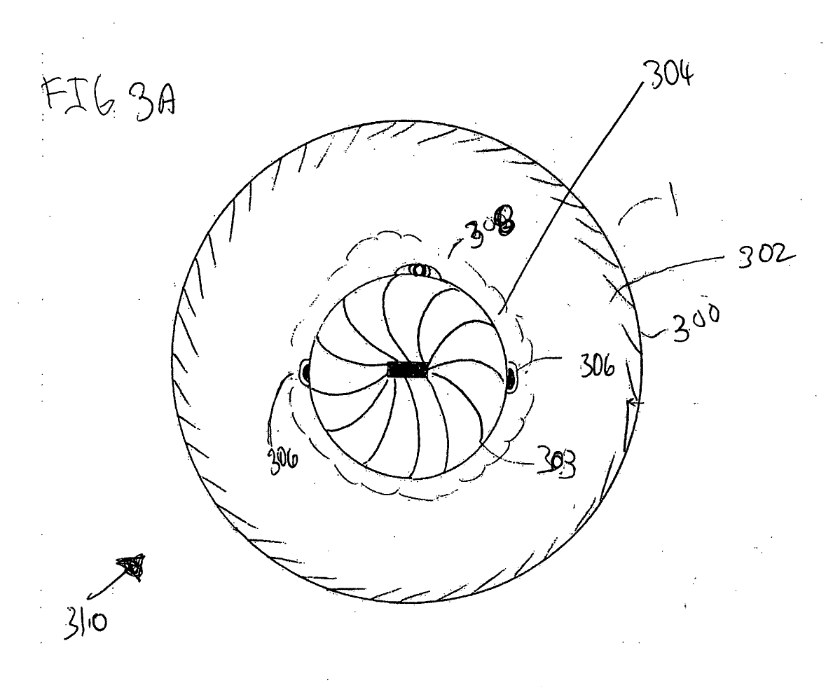

FIGS. 2A and 2B shows a detailed view of control area 4 depicted in FIG. 1A-B. Control unit 220 comprises control center 224, at least one or more fluid source containers 228. Preferably, control unit 220 further comprises at least one or more pumps (not shown). Optionally at least one or more pumps (not shown) is a fluid pump preferably able to create a fluid pressure between about 25 psig and to about 75 psig. Optionally, at least one or more pumps (not shown) is an air pump, preferably able to create a vacuum suitable for treatment according to the present invention. FIG. 2B provides a close up view of control center 224. Preferably, control center 224 comprises electronics as known and accepted in the optionally powered by a battery or mains power source (not shown). Preferably control center 224 comprises components as known and accepted in the art able to provide controllable vibrations preferably in the high frequency range, more preferably above about 25 kHz and below about 40 kHz, and most preferably in the frequency range of about 28 kHz to about 32 kHz. As shown in greater detail below, and also as previously described, optionally and preferably such vibrations are provided according to a piezoelectric effect.

Preferably, control center 224 comprises at least one or more display units 232. Preferably, display 232 provides an interface through which a user may interface, communicate and control the functioning of control center 224. Control center 224 may optionally comprise relatively simple electronics for providing such control or alternatively may comprise a processor, memory and so forth, for example as implemented through a computer, for such control. Optionally, display 232 provides a user with the ability of selecting a programmed treatment or customizing a treatment routine programmed into control unit 220. Preferably, control center 224 further comprises controls 235, 236 and 237 to control treatment parameters according to the present invention, for example including but not limited to vibration frequency, liquid delivery, liquid pressure, suction strength.

Control center 224 preferably features a frequency control switch 235 for the controlling the frequency of vibration used in the treatment protocol according to the present invention. Optionally and preferably the actions of control 235 is made visible on display 232.

Control center 224 also optionally and preferably features a suction control switch 236 for controlling the applied suction according to an optional treatment protocol of the present invention. Optionally and preferably the actions of control 235 is made visible on display 232.

Control center 224 also optionally and preferably features a fluid flow control switch 237 preferably for controlling the volume and/or pressure of fluid to be delivered according to an optional treatment protocol of the present invention. Optionally and preferably the actions of control 235 is made visible on display 232.

FIG. 3A shows a bottom view of an exemplary device according to FIG. 1A, specifically of the distal end of probe 1 having a probe face 310. A seal 300 is shown for sealing cover 302 to the area of skin to be treated (not shown; see FIG. 1A). Seal 300 may optionally be a silicon seal or any other type of sealing material as known and accepted in the art. Outlets 306 are shown, as is probe head 303. A suction outlet 308 is also shown, for providing suction, for example for applying a vacuum or alternatively for sucking out debris or liquid sprayed from outlets 306. Preferably sleeve 304 provides further compartmentalization or focus on the treatment point within the treatment area. Preferably sleeve 304 improve focus and visibility during treatment.

FIG. 3B shows another bottom view of another optional embodiment of the distal end of probe 1 having a probe face 320. As shown, suction 308 is placed at either side of the distal end of probe 1, while a plurality of outlets 306 are placed centrally, preferably arranged around probe head 303. Sleeve 304 and seal 300 are again shown as for FIG. 3A. Preferably sleeve 304 provides further compartmentalization or focus on the treatment point within the treatment area. Preferably sleeve 304 improve focus and visibility during treatment.

FIG. 3C shows yet another bottom view of another embodiment of the distal end of a probe having a probe face 330 according to an optional embodiment of the present invention. Again seal 300 is provided, which is maintained through a suction protruder 308. There is also preferably provided a plurality of internal seal units 332, each of which preferably comprises an internal suction/evacuation device 336 and an internal fluid spray outlet 334. Optionally individual fluid spray outlet 334 preferably operates in a pulsatile manner, more preferably according to manual control of the user (not shown) as depicted in FIG. 1A-B. Optionally individual suction device 336 then removes the sprayed fluid.

FIG. 3D shows another bottom view of another optional embodiment of the distal end of probe 1 having a probe face 340. Probe face 340 optionally comprises treatment head face 342 that lies within suction cup 300 that acts to seal the treatment area. FIG. 3E shows a close up view of treatment head face 342 in greater detail. As shown treatment head face 342 comprises sleeve 344, a plurality of suction nozzles 348 and a plurality of fluid jet nozzles 346, preferably arranged centrally around probe head 343.

Sleeve 304 and seal 300 are again shown as for FIG. 3A. Preferably sleeve 304 provides further compartmentalization or focus on the treatment point within the treatment area. Preferably sleeve 304 improve focus and visibility during treatment.

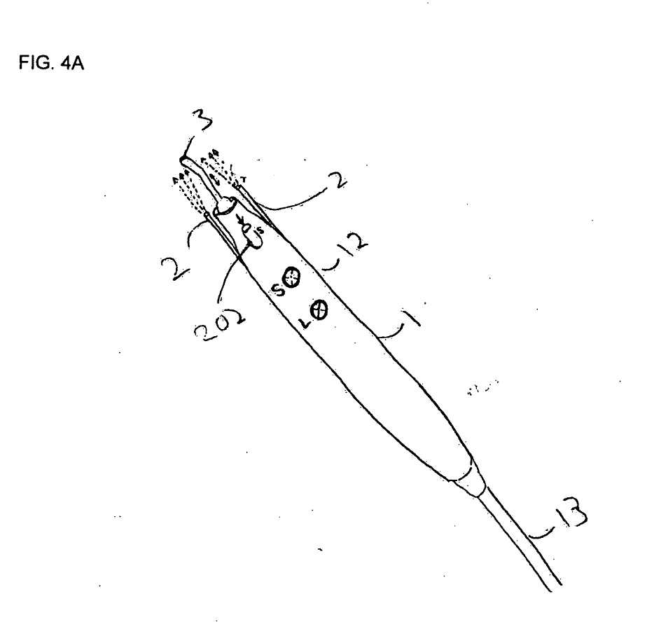

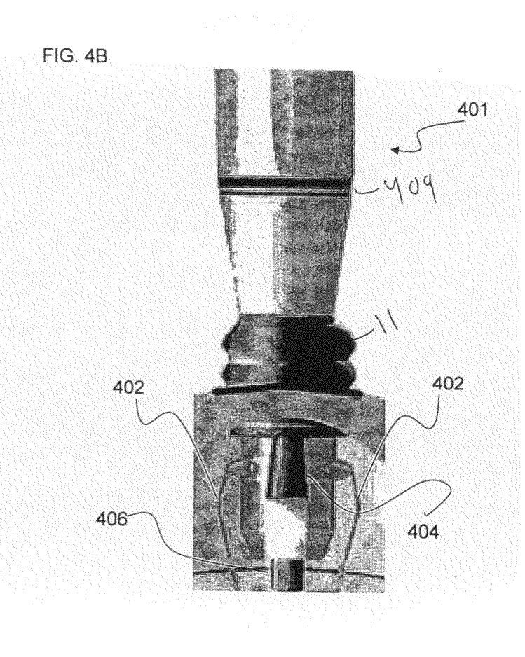

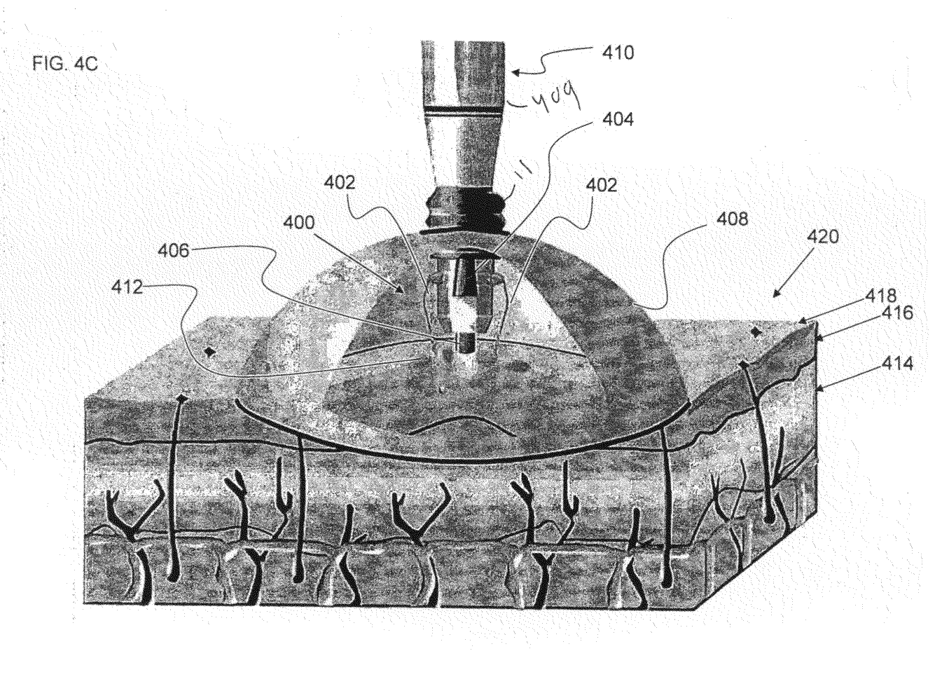

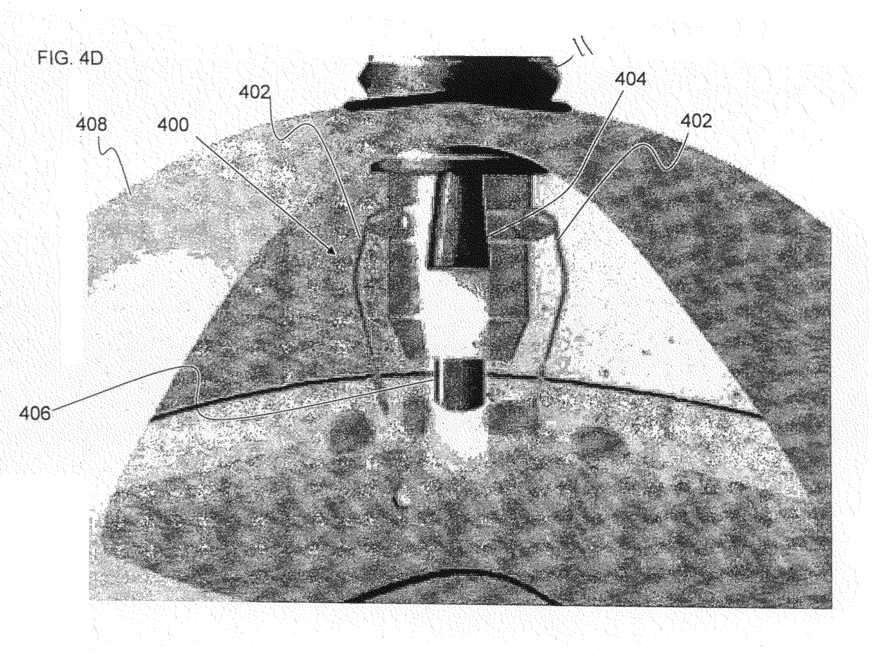

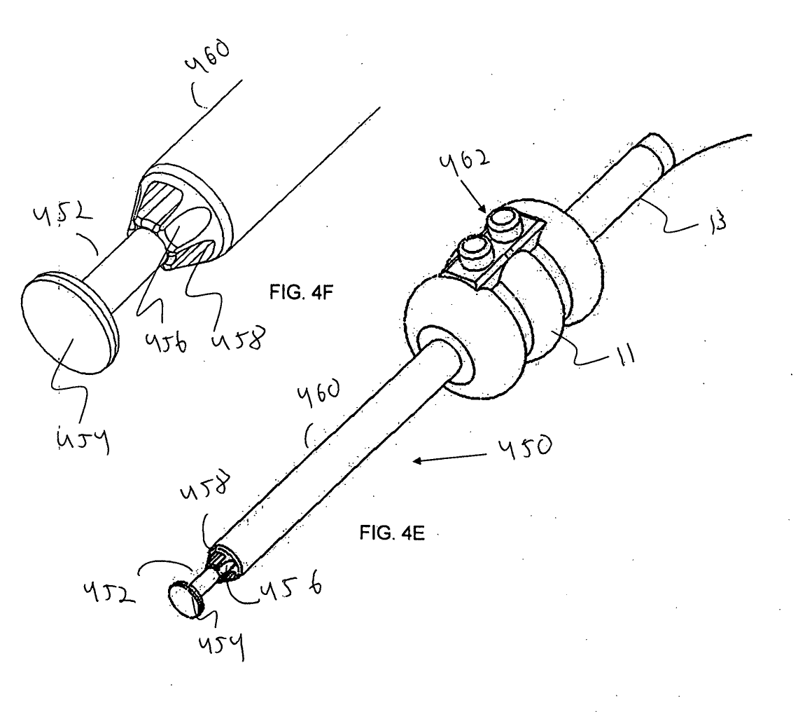

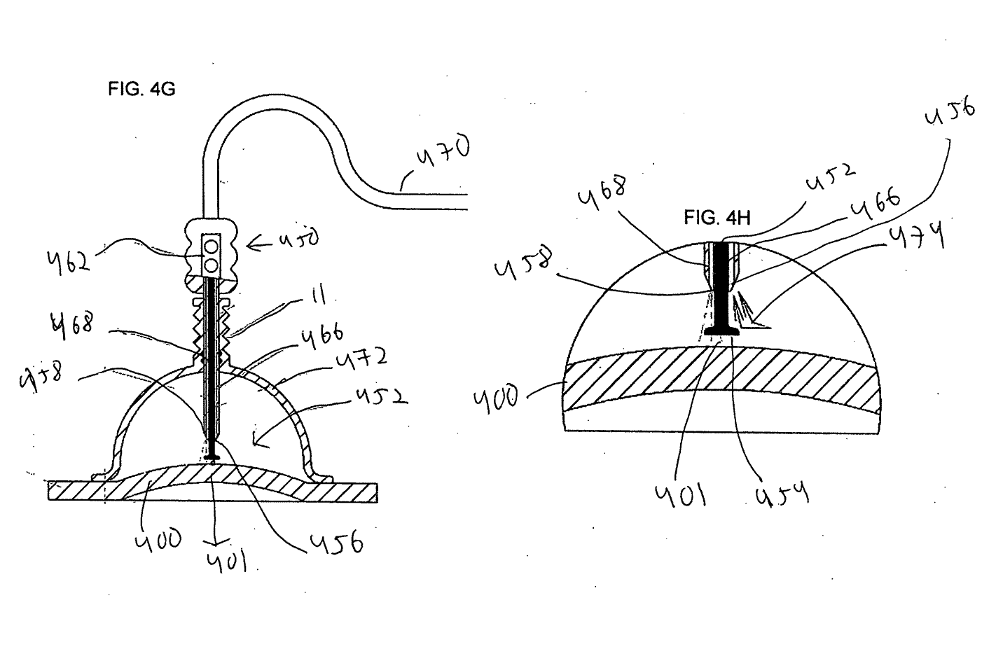

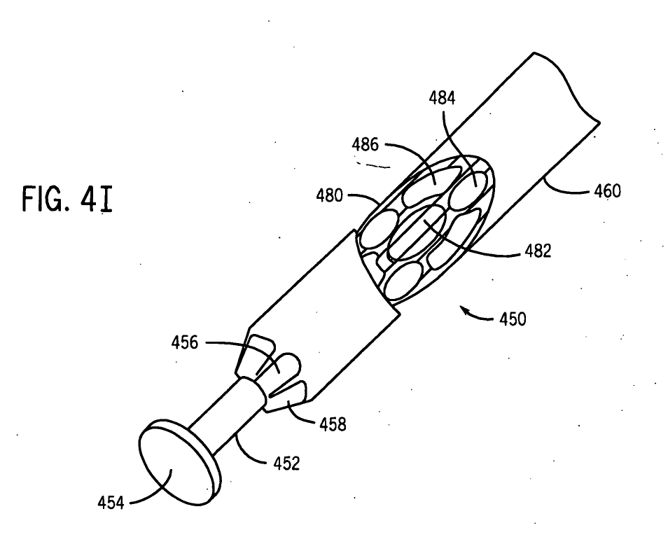

FIG. 4A shows an illustrative device according to the present invention, which may optionally be implemented with either of the systems shown in FIG. 1 or 2.

Probe 1 is again shown, with control buttons 12, connector 13 and probe head 3. Outlets 2 are optionally implemented as protruding nozzles as shown, rather than being implemented only as holes or openings. Suction 312 is also shown as optionally be implemented through a protruding tube. The operation of probe 1 may optionally be as described above.

FIG. 4B is a close up view of an optional embodiment of the distal end of probe 1 of FIG. 4A. Probe 401 preferably comprises a housing 409, flexible portion 11 (as previously described), jets 402, suction 404 and vibrating head 406.

FIG. 4C depicts an optional embodiment of the probe 410 according to the present invention to remove a tattoo over an area of skin 420. Skin area comprises dermis layer 414 above which lies the epidermis 416, above which lies the skin surface 418. The tattoo to be removed is incorporated within the dermis layer 414 in the form of ink 412. Probe 410 according to an optional embodiment of the present invention functions to displace ink 412 from the dermis 414 allowing it to be removed through the epidermis 416 and eventually out through the skin surface 418.

Probe 410 preferably comprises suction cup 408, a plurality of fluid jet nozzles 402, suction tube 404, and treatment head 406. Optionally probe 410 is between 5 cm to 20 cm long. Preferably, suction cup 408 optionally seals the treated skin area 400 preferably providing suction to the larger overall area being treated as well as the area specifically treated with treatment head 406. Preferably treatment head 406 is 1 mm to 20 mm in diameter. Suction tube 404 preferably provides suction over treatment area 400 within suction cup 408, preferably allowing treatment head 406 to lift the skin at the treatment point bringing the underlying ink 412 closer to treatment head 406. Preferably, treatment head 406 is vibrating at a frequency that is more than 25 kHz and lower than 40 kHz, most preferably between 28 kHz and 32 kHz, for example 30 kHz. Preferably while treatment head 406 treats treatment area 400 a fluid jet is applied the treatment area using a plurality of fluid jet nozzle 402. Optionally, fluid jet nozzle 402 delivers fluid at a pressure that is below about 75 psig and more preferably between about 25 psig and 70 psig.

FIG. 4D depicts a close up view of FIG. 4C showing the distal end of probe 410 and suction cup 408.

FIG. 4E depicts an optional embodiment of a probe 450 comprising a probe shaft 460, control buttons 462, and a probe distal treatment head 452. Treatment head 452 preferably comprises probe face 454, a plurality of suction tube nozzles 456 and a plurality of fluid jet nozzles 458. Optionally, suction tube nozzles 456 provides suction to the treatment area allowing a user to protrude the skin toward treatment head 452 and in particular to probe face 454, while also allowing a user to maintain a clean treatment area clear of debris. Most preferably, probe face 454 provides the treatment area with vibration frequency in the ultrasound range for example about 30 kHz. Most preferably vibration frequency is maintained within the range of about 28 kHz to about 32 kHz. Preferably, fluid jet nozzle 458 provides the treatment area with a fluid, preferably at a fluid pressure in the range of about 25 psig to about 75 psig, most preferably about 70 psig. FIG. 4F depicts a close up view of probe treatment head 452 wherein the plurality of suction nozzle 456, the plurality of fluid jet nozzle 458 and probe face 454 are more clearly visualized.

FIG. 4G depicts a sagittal sectional view of probe 450 of FIG. 4E applied to a treatment area 400. Treatment area 400 is defined by suction cup 472 comprising the distal end of probe 450 and treatment head 452. Optionally, suction cup 472 is made of a sealable material for example including but not limited to silicone, rubber or the like material known and accepted in the art able to create a vacuum over treatment area 400 that preferably protrudes treatment area 400 from underlying tissue (not shown) toward treatment head 452. Preferably, the point 401 lying directly beneath treatment head 452 receives the treatment according to an optional embodiment of the present invention. Preferably treatment head 452 vibrates treatment face 454 at a frequency between 28 kHz and 32 kHz while a plurality of fluid jet nozzle 458 provides a pressurized fluid delivered through a fluid tube 468 that is linked to control unit (not shown) similar to that depicted in FIG. 2 through tube 470. Preferably, treatment head 452 is further provided with suction originating at a control unit (not shown) via tube 470 that leads through to suction tube 466 and delivered to treatment point 401 through suction nozzle 456. Optionally, fluid delivery or suction delivery may be controlled with control buttons 462. FIG. 4H provides a close up view of the treatment area 400 and treatment point 401. Arrows 474 depicts the direction of fluid flow within treatment area 400. Fluid flow depicted by arrows 474 depict the optional air flow creating the vacuum within treatment area 400 or optionally depict the flow of a liquid fluid or debris away from treatment point 401 and treatment area 400.

FIG. 4I shows a close-up, partially cut-away view of probe 450 of FIG. 4E. As shown, removal (for the purposes of illustration only) of a portion of probe shaft 460 at cut-away 480 provides a view of internal components of probe 450. These internal components optionally include but are not limited to a rod 482 for transducing the vibrations from a piezoelectric material as previously described to treatment head 452. Rod 482 may optionally be constructed of any suitable material, such as a metal or metal composite for example. Preferably the material is selected so as to provide a compound resonator responsive to the frequency of vibrations of the piezoelectric material itself.

A tube 486, of which a plurality is preferably provided as shown, optionally and preferably supplies fluid to each fluid jet nozzle 458. A suction tube 488, of which a plurality is preferably provided as shown, optionally and preferably supplies force to each suction nozzle 456.

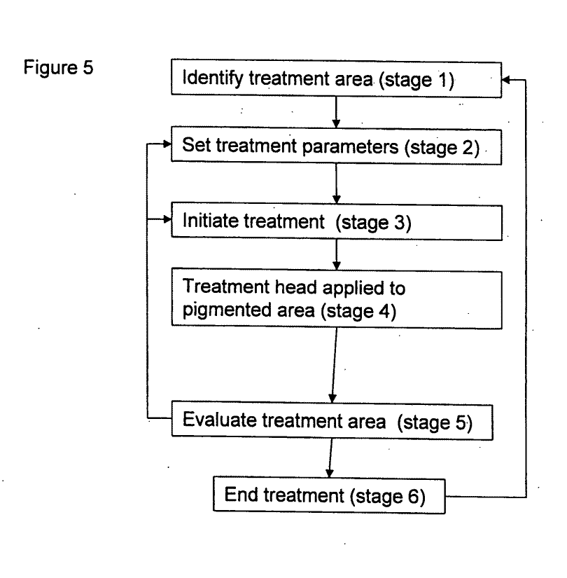

FIG. 5 depicts an optional method according to the present invention for pigment removal, for example for removing a tattoo. In stage 1 the area to be treated is identified. In stage 2 based on the parameters of the treatment the treatment parameters optionally including but not limited to vibration frequency, fluid jet pressure, and fluid solution are defined and loaded onto the system according to the present invention. In stage 3, the treatment is initiated over the treatment are optionally and preferably by placing the suction cup over it to create an appropriate seal and suction. In stage 4 the treatment head is applied to at least a portion but preferably the whole of the treatment area, optionally point by point such that the full pigmented area is treated uniformly. Optionally, the timing of fluid provision (whether pulsed, continuous or intermittent), vibrations and suction, and frequency thereof, is adjustable according to any combination of these forces and parameters. For example, fluid may optionally be provided with the vibrations, between application of vibrations, with or between application of suction, and so forth, more preferably as determined by the practitioner applying the treatment.

In stage 5 the treatment area is evaluated. Optionally at stage 5 the treatment is assessed wherein a plurality of options are preferably available. For example, treatment may be evaluated as being completed there for a user would proceed to end the treatment at stage 6. Optionally, the treatment may be reverted to stage 2 or stage 3 depending on the completeness of the applied treatment. For example, if the evaluation process reveals that the treatment parameters require adjustment, then they will be adjusted at stage 2 and continued from there. Optionally if the evaluation determined that the treatment was incomplete then the treatment would optionally be reinitiated at stage 3. At stage 6 the treatment is completed for the chosen treatment area. Optionally, a new treatment area may be evaluated, in which case the treatment method returns to stage 1 to evaluate the new treatment area.

### Experimental Example

The above described device, system and method of the present invention were tested experimentally in order to show their efficacy. The device was implemented according to a similar embodiment as for FIG. 1B. The objective was defined as testing the ability to perform complete removal of a tattoo (skin pigmentation/coloration) without damaging the skin. Prior to commencing the experiment successful removal was defined as complete removal of the mark without affecting skin texture.

The experimental procedure was approved and in accordance with the ethics committee on experimentation on animals (according to NIH standards), and with its prior approval.

### Materials and Methods

A male pig, of the large white type mixed with the Ladrance type, weighting 15 kg, was acclimated for 1 week following its arrival to the holding area.

### Food and Animal Care

The pig was fed twice daily with the commercially available product AMBAR and was allowed to drink freely ad-libitum.

### Anesthesia

The pig was anesthetized with a combined IM (intra-muscular) shot containing: 15 mg/kg body weight of Ketamine with 2 mg/kg body weight Xylazine. Following confirmation of complete anesthesia, an endotracheal tube was inserted into trachea of diameter number 6. The outer portion of the pipe was connected to an automatic gas anesthesia machine using 2%-3% Isoflurane mixed with oxygen.

### Creation of Skin Pigmentation

A hand held animal tattoo device was used to create the pigmentation. Blue ink was inserted into the depths of the dermis layer with a special needle. Tattoos were created in 8 locations along either side of the pig's back.

The pig was disconnected from the gas anesthesia machine and allowed to recover in the holding pen. The pig was kept in recovery for a period of 6 weeks following the procedure, allowing enough time, according to reports in the literature, for the pigment to sufficiently associate with macrophage cells in the tissue (which is believed to be the mechanism through which the pigment is permanently maintained in the skin).

### Initial Treatment Procedure

Following 6 weeks the pig was anesthetized once more following the same protocol. The device of the present invention was used on 6 of the 8 locations tattooed. A solution containing 2% salicylic acid was used, although optionally another material such as glycolic acid, lactic acid, or a combination thereof may be used. This low concentration of salicylic acid was used because it is described in the literature as being useful for cleaning the skin in sensitive areas and does not drastically damage the skin. As a precautionary method, the antibiotic Cefazolin 750 mg was administered intravenously, to prevent any potential infection. The pig was then returned to his pen and observed for a 3 week period. The second treatment procedure was done undertaken following the skin's full recovery.

### Secondary Treatment Procedure

Following 3.5 weeks from the first procedure, the pig was anesthetized as described above. The device was used on 4 tattoo sites alone. At this stage antibiotics were not used.

The pig was returned to the pen and follow up observation was performed including photographs of the treated sites, from both the initial and secondary procedures.

### End of the Experiment

Following euthanasia all 8 procedural sites were excised and taken for histology/pathological testing. For euthanasia, the pig was first anesthetized as described above followed by an IV injection of Pentobarbitone Sodium 135 mg/kg of body weight.

### Results and Conclusions

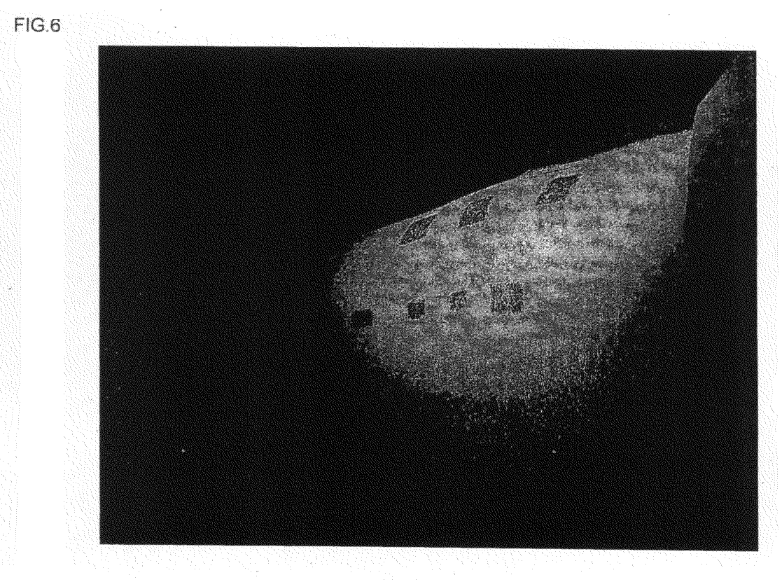

Of the eight tattooed sites, six were treated while two served as controls. Two of the treated sites underwent the initial procedure alone, while the remaining four sites were treated with the second (and final) treatment. The treatment was conducted with vibrations at the rate of about 30 kHz; the water was administered at between 25 to 70 psig. FIG. 6 shows all eight sites after tattooing (introduction of pigment) but before treatment.

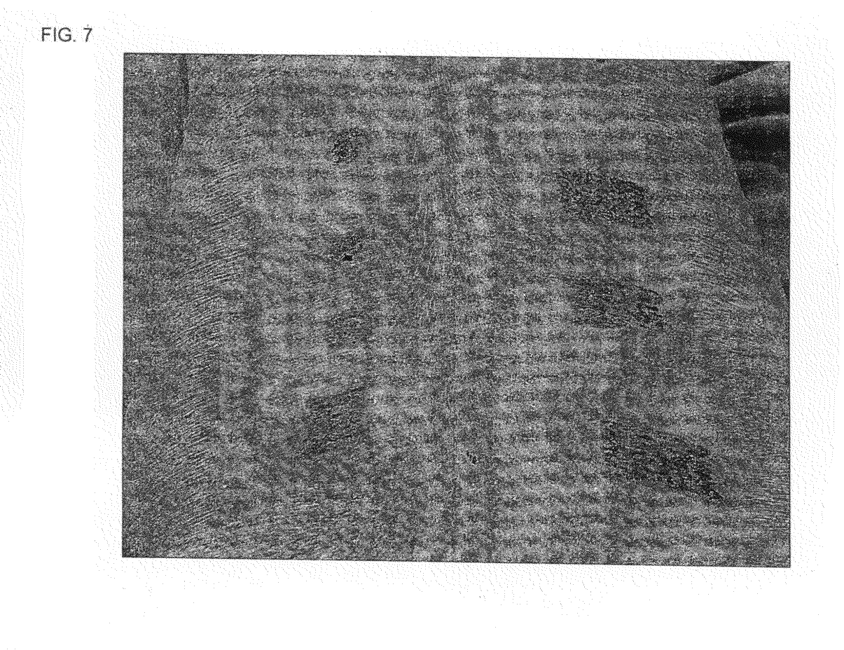

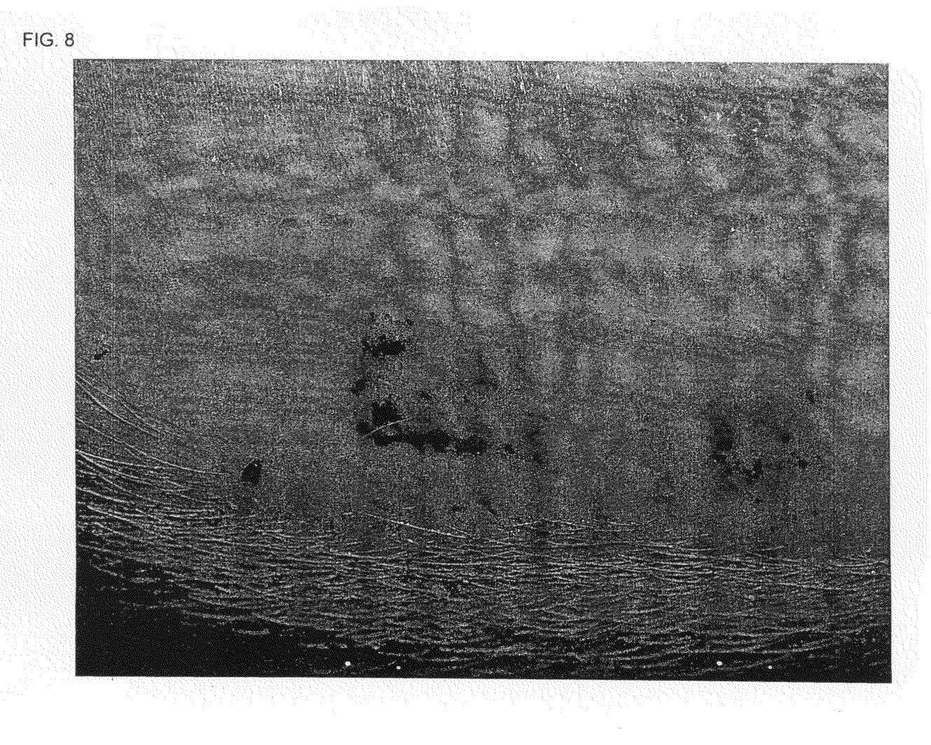

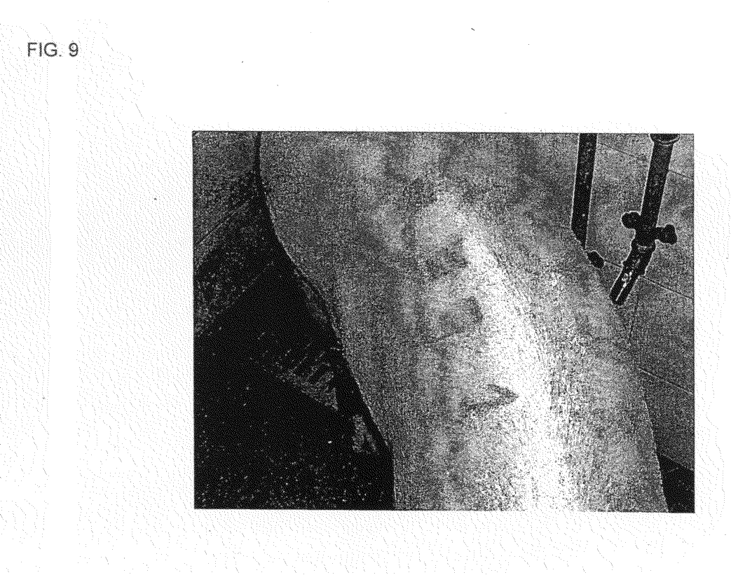

Following one or two treatments, changes in skin texture were not observed. No indication of scarring was found. No permanent damage was caused to the epidermis; any damage seen was temporary and was completely regenerated. FIG. 7 shows four sites after one treatment (the four treated sites appear on the left). FIG. 8 shows two of the four sites after two treatments. FIG. 9 shows the entire area of skin after the second treatment was applied; the four sites on the right were treated twice and show a nearly complete eradication of the pigment. On the right, the bottom two sites (including one square and the number “one”) were not treated, while the top two sites were treated once.

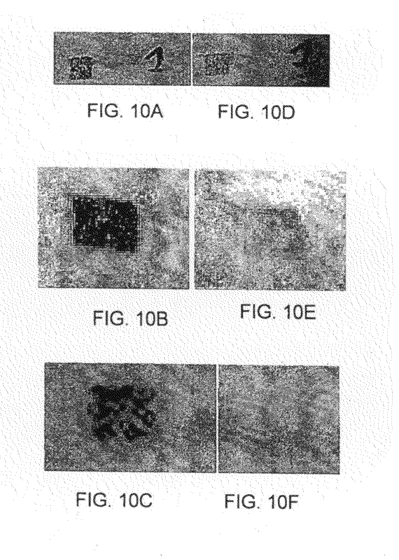

After initial treatment alone, separation of pigment from the macrophage cells removal was estimated to be 40%, as 40% of the pigment was estimated to have been removed. Following the second treatment, removal was estimated at 70%-90%. FIG. 10 shows a comparative look at before and after photographs following treatment at different states. FIG. 10A depicts photographs of tattoos in the control group before treatment while FIG. 10D depicts the control group's tattoo following treatment. FIG. 10B is a photograph of one of the tattoos prior to treatment, while FIG. 10E depicts the same tattoo following a first treatment according to the present invention, resulting in about 30% to 40% pigmentation removal. FIG. 10C is a photograph of one of the tattoos prior to any treatment, while FIG. 10E depicts the same tattoo following a second treatment according to the present invention, resulting in about 80% to 90% removal of the original tattoo.

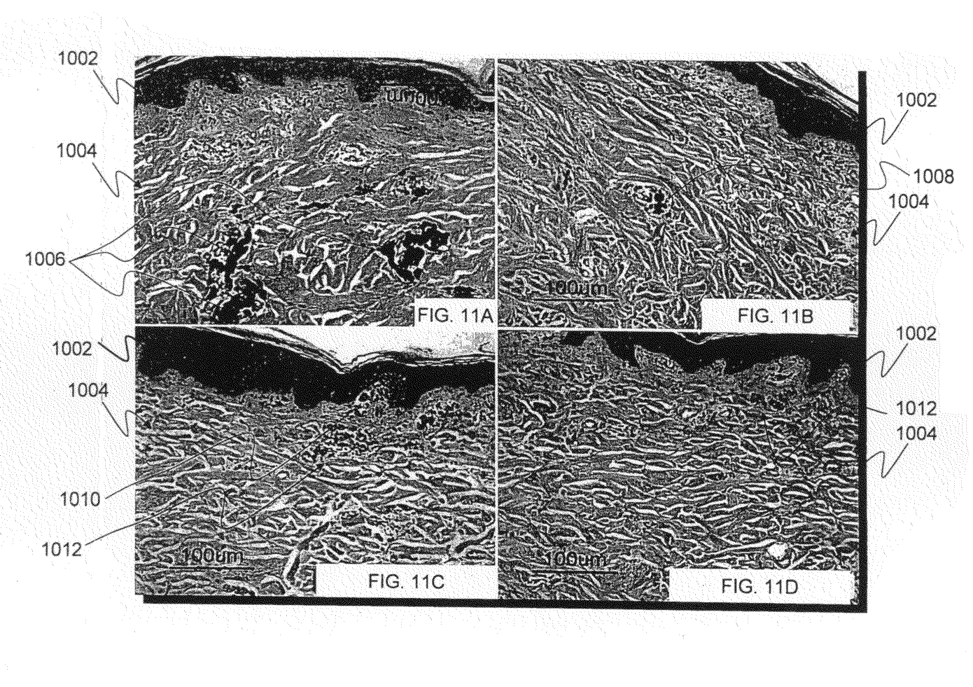

FIG. 11 further shows the results of the histological study following the end of the experiment. FIG. 11A shows a histological section of the control group prior to treatment according to the present invention. The epidermis layer 1002 and the dermis layer 1004 where the ink 1006 is embedded within dermis layer 1004 below the epidermis 1002.

FIG. 11B depicts a histological section following a single treatment according to the present invention, 1008 depicts the remaining ink within the dermis 1004. FIGS. 11C and 11D show the epidermis 1002 and dermis 1004 following a second treatment according to the present invention where the amount of ink 1012 is reduced and dispersed closer to the transition area between the dermis 1004 and epidermis.

Thus, it can be seen that the non invasive procedure according to the present invention does not affect the skin texture, and causes extensive and efficient removal of the pigment in a short period of time with very high efficiency, minimal damage to the skin and also rapid recovery.

While the invention has been described with respect to a limited number of embodiments, it will be appreciated that many variations, modifications and other applications of the invention may be made.

1.-55. (canceled)

56. A system for removing pigmentation from skin, comprising:
a. a container for containing fluid to be applied to the skin;
b. a pump fluidly connected to said container for pumping said fluid from the container under pressure; and
c. a probe fluidly connected to said container for received pumped fluid, said probe comprising an outlet at a distal end of said probe for spraying said fluid onto the skin, said distal end vibrating to apply vibrations to the skin.

57. The system of claim 56, further comprising a skin protruder for causing the skin to protrude at an area being sprayed with said fluid.

58. The system of claim 56 wherein said probe has a distal end having a diameter between 1 mm to 20 mm.

59. The system of claim 56 wherein said probe vibrates at a high frequency in the range of from about 25 kHz to about 40 kHz.

60. The system of claim 56 further comprising a suction cup comprising the distal end of said probe.

61. The system of claim 60 wherein said suction cup and probe distal end are assembled into a single interchangeable unit.

62. The system of claim 56 able to control parameter chosen from the group comprising fluid temperature, fluid pressure, fluid volume, fluid direction and fluid rate.

63. The system of claim 56 further comprising a control center to control system functions.

64. A device for removing pigmentation from skin through the application of fluid, comprising: a probe for receiving the fluid, said probe comprising an outlet for spraying the fluid onto the skin.

65. The device of claim 64 wherein said probe has a distal end having a diameter between mm to 20 mm.

66. The device of claim 64 wherein said probe vibrates at a high frequency in the range of from about 25 kHz to about 40 kHz.

67. The device of claim 64 further comprising a suction cup comprising the distal end of said probe.

68. The device of claim 67 wherein said suction cup and probe distal end are assembled into a single interchangeable unit.

69. A method for removing pigmentation from skin, comprising:
Applying vibrations to the skin; and
Applying fluid non-invasively to the skin.

70. The method of claim 69, wherein the fluid comprises a drug or treatment material.

71. The method of claim 69, wherein the vibrations are at a frequency between about 25 kHz and 40 kHz.

72. The method of claim 69, wherein the fluid pressure is at 25 psig to 75 psig.

73. The method of claim 70, wherein the vibrations are at a frequency between about 25 kHz and 40 kHz.

74. The method of claim 70, wherein the fluid pressure is at 25 psig to 75 psig.

75. The method of claim 71, wherein the fluid pressure is at 25 psig to 75 psig.
